(19) United States
(12) Patent Application Publication
(10) Pub. No.: US 2009/0048506 A1
(43) Pub. Date: Feb. 19, 2009

(54) METHOD AND SYSTEM FOR ASSESSING BRAIN FUNCTION USING FUNCTIONAL MAGNETIC RESONANCE IMAGING

(76) Inventors: Alina K. Fong-Ichimura, Murray, UT (US); Mark Allen, Provo, UT (US)

Correspondence Address: BATEMAN IP LAW GROUP, P.O. BOX 1319, SALT LAKE CITY, UT, 84110, US

(21) Appl. No.: 12/190,566

(22) Filed: Aug. 12, 2008

Related U.S. Application Data
(60) Provisional application No. 60/955,577, filed on Aug. 13, 2007.

(51) Int. Cl. A61B 5/055

(52) U.S. Cl. 600/411

(57) ABSTRACT

A system for assessing brain functioning uses neuropsychological assessments which are performed while a patient is within an fMRI scanning environment and while scanning is in process to detect the BOLD signals within the patient's brain. The system may be used to detect brain impairment and injuries which do not present tissue damage detectable using previous methods.

### RELATED APPLICATIONS

The present application claims the benefit of U.S. Provisional Application Ser. No. 60/955,577, filed Aug. 13, 2007, which is expressly incorporated herein in its entirety by reference.

### BACKGROUND OF THE INVENTION

1. The Field of the Invention

The present invention relates to a method and system for assessing brain functionality. More specifically, the present invention relates to a method for utilizing magnetic resonance imaging to monitor brain activity in order to assess brain functionality and damage to the brain.

2. State of the Art

Magnetic resonance imaging (MRI) is used to detect tissue damage. MRI technology is important to the medical field because, while X-ray or CT imaging are more sensitive to detection of bone or hard tissues, MRI is sensitive to the soft tissues of the body and can differentiate between different adjacent tissues. Thus, MRI has been used to detect tissue damage by detecting the differences between damaged and healthy tissue, such as the increase in fluids often present in a damaged tissue

Clinical neuropsychology is a specialty of psychology which specializes in the assessment and treatment of patients with brain injury or neurocognitive deficits. Neuropsychologists have used brain-behavior relationships to determine whether impairment is present. The neuropsychologist will test skills such as reasoning, learning/recall, attention/concentration, perception, sensation, language processes, and controlled/directed movement to determine the person's ability to perform these skills, and thus determine impairment.

In order to accurately and consistently determine impairment, a number of neuropsychological tests and assessments have been developed which are designed to evaluate the cognitive abilities of a person, and thus determine the presence and degree of impairment. These tests may include memory assessment scales, verbal learning tests, continuous performance task, decision making tests, sorting tests, etc. The tests are standardized and statistical information for the test performance of a large number of persons is typically available. As such, the tests are designed to allow the treating neuropsychologist to observe the performance of the patient in taking the test and compare the patient's performance with that of the general population to determine if the person has impaired brain functioning for the tested skills.

While useful in detecting and assessing brain injury or mental impairment, the existing tests and methods have drawbacks. MRI scans, for example, are useful in detecting unusual tissues such as tumors or scars or tissues which show edema or other structural damage to the tissue. Conventional MRI scans, however, are not useful in detecting impaired brain functionality where the brain tissues do not manifest physical damage.

The standard paper and pencil neuropsychological tests used to determine a person's cognitive abilities, and thus to determine if any impairment is present, are also limited in their abilities to properly diagnose brain impairment. The tests used for evaluation do not measure brain functionality, but measure the ability to perform predetermined tasks and brain functionality is inferred therefrom. As such, the tests are not able to determine if a low level of performance or cognitive ability is the result of an injury, or is simply the result of a lack or education or experience or even the result of a person's deliberate attempt to achieve a low score in order to receive a benefit such as a disability benefit or medical leave. Additionally, these tests rely on human perception of performance and determination of any impairment, and are thus subject to human errors.

It would be desirable in many cases to directly measure brain functionality instead of observing a person's abilities to therefrom infer brain functionality. Such may be the case where a worker is claiming an injury affecting the brain and requesting benefits or compensation, where a soldier is claiming a battle injury or shell shock, etc. has caused impairment. In these cases, persons may be motivated to achieve low testing scores as they desire to receive a benefit. In such a situation the ability to measure and observe the brain functionality instead of simply observing the performance of tasks may allow the treating neuropsychologist to better determine if brain injury and impairment is actually present.

It would also be desirable to directly measure brain functionality where a person is being treated for an injury or impairment. Typically, the purpose of diagnosing and assessing a brain injury or impairment is to provide a treatment suited to correcting the condition. The treatment may involve surgery, drugs, physical or mental exercises, etc. Where a person is being treated, it is typically desirable to determine whether the treatment is effective or not. It would thus be desirable to be able to directly monitor brain functioning before and during or after the treatment to determine the effectiveness of the treatment. Similarly, it would be desirable in a variety of situations to periodically monitor a person's brain functionality in order to track improvement or deterioration of brain functionality.

It is thus desirable to have a system and testing methods which allows direct observation of a patient's brain functions instead of only allowing observation of the patients ability to perform a task.

### SUMMARY OF THE INVENTION

It is an object of the present invention to provide an improved method and system for assessing brain function using imaging of the brain.

According to one aspect of the invention, functional MRI scans (fMRI) may be taken of a patient's brain while the patient is performing designated tasks in order to assess the level of brain function. The use of fMRI scans allows a person to observe small changes in oxygen flow that occur throughout the brain due to fluctuating activity levels. The fMRI scans allow a person to directly observe activity levels in a brain.

According to another aspect of the invention, a series of neuropsychological assessment tests which could be performed while a person is in an MRI scanning environment are provided. The neuropsychological assessments are performed while a person is positioned in an MRI machine and while fMRI scans are taken of the person's brain.

According to another aspect of the invention, a normative data set of brain activity is created, allowing the correlation of normal brain activity to assessment test task performance. The normative data set and correlation to the assessment tests results in probabilistic activation maps corresponding to the neuropsychological assessment tests. Such probabilistic activation maps allow for comparison of the brain activities of a patient with those of the normal population to determine if an impairment is present in the patient.

These and other aspects of the present invention are realized in a method and system for assessing brain function using functional magnetic resonance imaging as shown and described in the following figures and related description.

### BRIEF DESCRIPTION OF THE DRAWINGS

Various embodiments of the present invention are shown and described in reference to the numbered drawings wherein:

FIG. 1 shows side and top views of a brain indicating scanning grids as used in the present invention;

FIG. 2 shows a theoretical BOLD signal resulting from brief neural activity in a brain;

FIG. 3 shows a theoretical BOLD signal resulting from sustained neural activity in a brain;

FIG. 4 shows a test procedure according to the present invention and illustrates idealized periods of brain activity;

FIG. 5 shows test procedure of FIG. 4 conformed to the BOLD signal of FIGS. 2 and 3 to represent a theoretical BOLD signal resulting from the testing periods of FIG. 4;

FIG. 6 shows a design matrix for a patient utilizing the theoretical signal of FIG. 5 and compensating for other signals generated in the brain;

FIG. 7 shows a map of t-values corresponding to a patient as may be used to generate a probability map of the patient's brain;

FIG. 8 shows a sample screed of a matrix reasoning test of the present invention;

FIGS. 9-14 show time sequences and sample screens for neuropsychological assessment tests of the present invention;

FIGS. 15-20 show theoretical areas of brain activity corresponding to BOLD signal as is expected to be generated by the neuropsychological assessment tests of the present invention overlaid on generalized fMRI scanning slices of a brain;

FIGS. 21-26 show measured areas of brain activity corresponding to a BOLD signal as is actually generated by the neuropsychological assessment tests of the present invention; and

FIG. 27 shows a scanning system according to the present invention.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as such, multiple figures are presented to separately illustrate the various details of the invention in greater clarity. Similarly, not every embodiment need accomplish all advantages of the present invention.

### DETAILED DESCRIPTION

The invention and accompanying drawings will now be discussed in reference to the numerals provided therein so as to enable one skilled in the art to practice the present invention. The drawings and descriptions are exemplary of various aspects of the invention and are not intended to narrow the scope of the appended claims.

MRI technology has provided static cross-sectional images of tissues such as brain tissues. MRI images are useful in detecting abnormalities in tissue such as tumors or damaged tissue. MRI technology has had limited use in detecting impairments of brain functionality as many injuries to the brain which result in impaired functionality are not easily visible on an MRI scan. Recently, functional MRI scans have been developed. These fMRI scans are taken over a period of time and can be used to identify changes in the tissue which occurred during that time.

The present invention provides a series of neuropsychological assessments which may be performed within a MRI machine while a fMRI is being taken and also provides probabilistic activation maps of the brain, i.e. normal brain response, which is correlated to the assessments and which may be used to determine whether a person's particular brain response while engaged in the tasks of the assessments is within the normal range of brain response or activity, or indicates damage to a particular area of the brain of otherwise indicates an impairment. The present invention provides a method and system for using fMRI to diagnosis and assess impairment.

According to the present method of diagnosis and assessment, a patient will undergo a series of fMRI scanning sessions; each session involving various tasks designed to assess brain functionality. Currently, six assessment tests have been designed. It will be appreciated that not every assessment test/scanning session will be necessary in every situation. In each session, the patient will perform a task or series of tasks while continuous fMRI scans covering the whole brain or a relevant part of the brain are collected. The tasks for each session are standardized so as to be performed as a standard and uniform test, providing uniformity of results and allowing the comparison of the patient's particular results (resulting brain fMRI scans) with those of a database of results for normal individuals to determine if an impairment is present.

In order to diagnose and assess normal brain function versus an impairment, it is desirable to identify the areas of the brain which are active during a particular test and to compare the fMRI scans of those areas when comparing the patient results with the normative data.

In operating the MRI device to achieve the desired fMRI images, the device was operated using a gradient echo, echo-planar imaging (EPI) sequence. This EPI protocol is optimized for detecting subtle changes in blood oxygenation levels in the brain over time. EPI scanning is thus an effective way to measure changes in the Blood Oxygen Level Dependent (BOLD) signal which has been shown to reliably correlate to changes in neural activity. Scans were taken which covered the whole brain, allowing the continuous monitoring of the whole brain throughout each assessment test/scanning session.

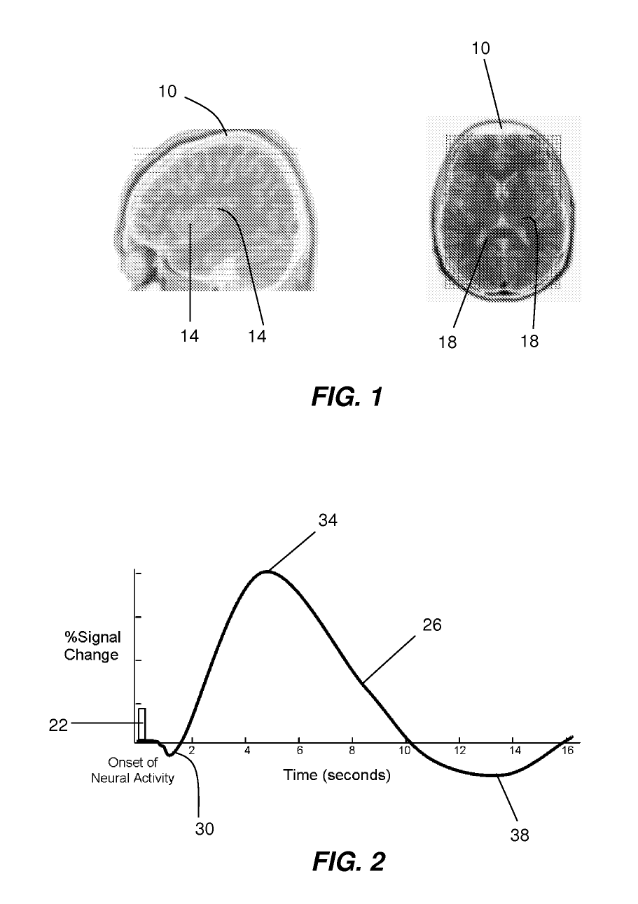

The BOLD scans were taken along 23 contiguous horizontal (axial) slices in a bottom-up, interleaved fashion. Each axial slice was 5 mm thick and was divided into a grid of 64 by 64 sections, resulting in 3.75 mm by 3.75 mm sections. Thus, the brain was sampled in voxels (three dimensional pixels or sample cells) which are 3.75 mm by 3.75 mm by 5 mm. The EPI sequence required 2 seconds for each whole brain image. The brain was thus sampled continuously to achieve a complete scan every 2 seconds. FIG. 1 shows a human brain 10 and the placement of the axial slices 14 and the grid of sample cells 18 within each slice.

In establishing a series of neuropsychological assessments for use with fMRI scanning and for correlating the results of the scans with the testing procedures, it is important to understand the contribution of the hemodynamic response to neural activity (the changes in blood flow such as capillary expansion which accompany neural activity), as it is the most significant contributor to the BOLD signal measurable in the fMRI scans. FIG. 2 shows a typical change in the BOLD signal over time for a cognitive event 22 which happens very quickly, such as recognition of a letter or object. As illustrated in FIG. 2, the resulting BOLD signal 26 may have a slight initial dip 30, and begins to rise about two seconds after the onset of neural activity 22. The BOLD signal 26 typically peaks 34 between four to six seconds after the onset of neural activity, and returns to baseline about ten seconds after the onset of neural activity. The BOLD signal 26 may dip below the baseline 38 between ten and fifteen seconds after the onset of neural activity.

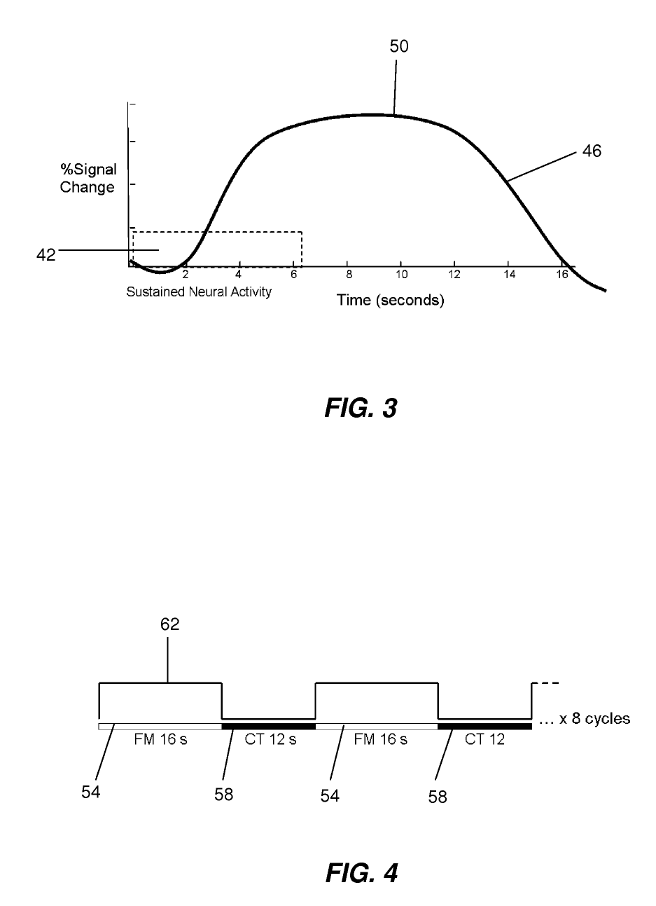

FIG. 3 shows a typical change in the BOLD signal 46 over time for a cognitive event 42 which is sustained over a period of time, such as verbal rehearsal or complex problem solving. The sustained cognitive activity will result in a sustained peak in the BOLD signal 46. The BOLD signal is similar to that of a brief cognitive event as shown in FIG. 2, but has an extended region of peak activity 50. The peak 50 in the BOLD signal 46 is again reached about four to six seconds after the onset of neural activity and is extended for a time period approximately equal to the duration of the neural activity 42. After the end of the peak signal 50, the BOLD signal 46 returns to baseline and may dip below baseline as shown in FIG. 2.

Typically, a BOLD signal 46 with a sustained peak 50 will be easier to detect and measure than a BOLD signal 26 with a brief peak 34, as the BOLD signal may often be affected by cardiac and respiratory cycles, magnetic fluctuations, atypical HRF response or coupling, atypical metabolic processes, etc. BOLD signals 26 with brief peaks 34 (corresponding to fast cognitive events 22) may be made easier to detect by repeating the event 22 and by employing tight control over the testing procedures (i.e. the application of the neuropsychological assessments).

A strategy for administering the psychological assessments of the present invention may therefore be to administer the assessments to a patient in a sustained manner (continuous or sequenced/repeated tests) for a period of ten to eighteen seconds and then provide a rest for ten to eighteen seconds to prevent fatigue or acclimation to the testing procedures with a subsequent reduction in the BOLD signal. It is typically important to control the patient's behavior in the rest periods. If a patient is provided with little or no direction as to what they should do during a rest period (such as providing no direction or stimulus, or providing a non-demanding task such as looking at a cross or other simple object), the patient will typically begin to day-dream, reminisce, plan future events, ponder problems, etc. These activities are cognitive processes such as covert language, memory, and executive functioning, and may interfere with the desired cognitive assessment. As such, a patient may be required to count during rest periods in order to provide a simple yet structured task.

The creation of a test which provides control and direction both during the actual testing periods as well as the resting periods in between will provide more accurate test results. The test results may be more accurate in different ways. The measured changes in the BOLD signals detected by the fMRI scans may be more accurate in amplitude, providing better comparison with the normal population and more accurately assessing the presence and extent of an impairment. Additionally, the control of the resting periods between testing sessions, as well as the consistency in applying the test at accurate predetermined time intervals, leads to results which are more accurate and consistent in terms of the starting times, stopping times, and peak signal times and amplitudes.

The fMRI testing environment is a relatively tiresome environment. It is fairly noisy, the person is not allowed to move during the test, and even talking will typically result in sufficient movement to affect the test results. The present invention thus provides tests which provide for successful neuropsychological assessment in the fMRI environment. The assessments are carried out without requiring the use of metal equipment or other objects which are problematic in the fMRI environment. The assessments are also carried out in a way that does not require patient movement or talking. The assessments are also designed to provide structure and control so as to keep the patient focused on the test without becoming exhausted and thereby provide consistent results.

The statistical approach used to evaluate the general set of fMRI data is a time-series variant of the Analysis of Covariance (ANCOVA) form of the general linear model. The statistical analysis tests each voxel of the brain, for each subject, against the null hypothesis (that over the duration of the testing the rise and fall of the BOLD signal coming from that voxel does not correlate with the onsets and offsets of the cycles of cognitive tasks). A weighted model is created that begins with a simple square wave type model of the on-off timing events for a single task variable of interest. Such an approximation is shown in FIG. 4. The alternating testing periods 54 and resting periods 58 (which may be counting or another controlled activity) will generally produce a simplified output signal as indicated at 62.

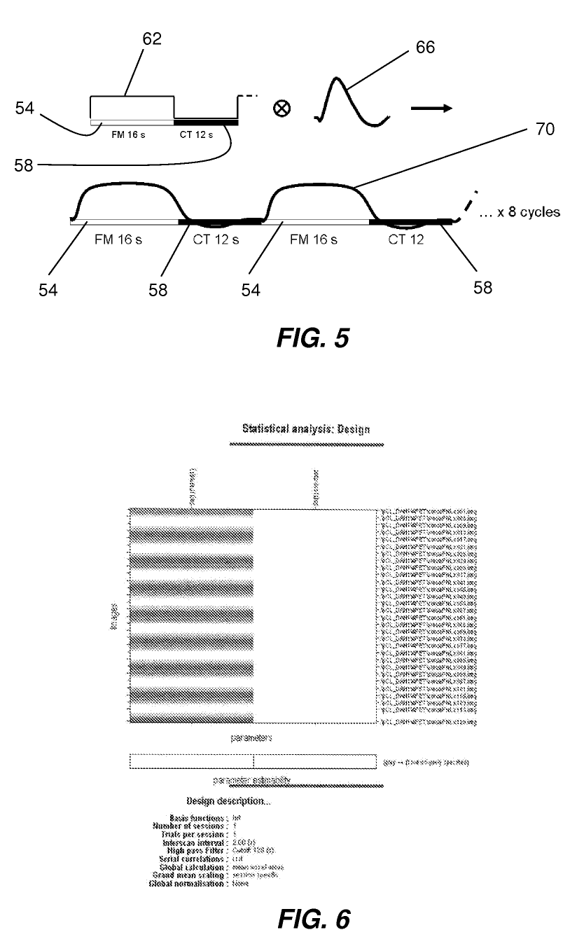

As the BOLD signal is expected to follow the response curve as discussed and shown in FIGS. 2 and 3, the simplified signal indicated at 62 is convolved with the BOLD response curve 66, as shown in FIG. 5. The result is the expected BOLD signal output (that measured by the fMRI scans) as indicated at 70.

The ANCOVA model allows regressors to be used to model nuisance variables. In the present invention, the global signal strength of the whole brain is used as such a regressor to isolate the BOLD signals produced as a result of the psychological assessment. The global signal strength of the brain will account for fluctuations in the brain caused by respiratory cycles, cardiac flow cycles, etc. These signal variations occur across the whole brain, and may often be a greater magnitude than the localized changes in the BOLD signal resulting from the assessments. These signal variations occurring across the brain can be subtracted from the measured fMRI signals to show the signals resulting from the assessment test.

An example of the complete ANCOVA model for a single subject with one covariate (an assessment test, here Face Memory) and one regressor (Global Signal) is shown in FIG. 6. FIG. 6 illustrates a design matrix, and is generated by analysis of the fMRI results. The FM covariate is represented by the left column, and the Global regressor is on the right. Time is represented on the y-axis, in a top-down fashion. The weight strength, at each time point, is represented in terms of grey-level brightness, with the strongest positive weighting represented as the brightest white, and the lowest (negative) weighting as darkest black. Thus, the brightest points correspond to the peaks of the HRF curve of FIG. 5 and the dark regions correspond to the low points of the curve.

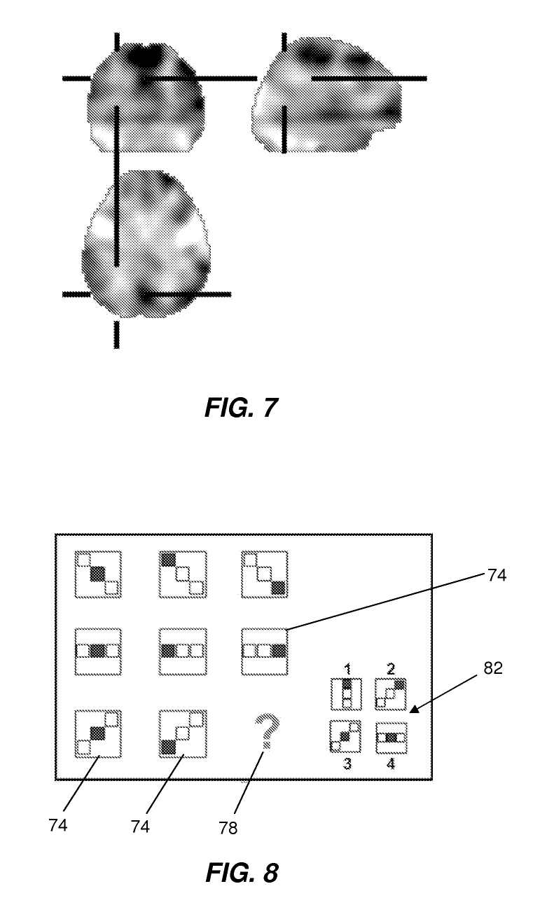

The resulting product of the ANCOVA computation on a single-person's fMRI data is a 3-dimensional map of t-values, as shown in FIG. 7, which illustrates slices of the person's t-value map. The magnitude of the t-value at each voxel is represented as grey-level brightness, with brighter voxels representing higher t-values.

A t-value map can then be converted into a probability map (a map of corresponding p-values) and the results can be displayed at whatever threshold is desired. (e.g., p<0.05). The results may be overlain on a higher-resolution MRI image, in order to facilitate identification of finer-grained cortical structures.

In order to analyze data from more than one subject, each subject's brain is first normalized into a common 3-dimensional stereotactic space before each individual's t-map is computed. Then the value of the sum of the contrast weights for each voxel from each subject computed during the ANCOVA (basically, the numerator of the t-statistic) is entered as a single data point in a new, “second-level” t-statistic computation. In this second-level computation, then, the mean value for each voxel across subjects is modeled as the effect term and the variance between subjects as the error term. An important consequence of this approach to keep in mind is that it is very unlikely that a voxel will show significant activation on the group-level map, unless virtually all of the subjects show activation at that voxel.

In order to make the statistical data and analysis methods relevant to the field of neuropsychology, it is desirable to provide participants with a testing experience that corresponds to what a person experiences in standard paper and pencil neuropsychological evaluation. However, the MRI scanning environment itself in many ways prohibits exact replications of neuropsychological assessments. One major challenge, therefore, was to approximate as closely as possible the standard test battery used for neuropsychological evaluation within several confines and limitations of fMRI scanning without deviating so far from the basic format of the assessment as to render the results incomparable with traditional testing methods.

The significant limitations of the MRI scanning environment include: 1) the subject is not able to move, 2) there can be no metal in the scanning room, 3) the task needs to be repeated several times, 4) sessions must be fairly short (<5 minutes), or else data files become too large to handle computationally. The various types of psychological assessment tests are adapted to a MRI scanning environment as follows:

In order to provide assessment protocols which may be performed within a MRI scanning environment, a projector is used to present the assessment tests to the person, and the person is provided with a fiber-optic response pad which detects when the person presses the pad and has therefore finished with the task at hand. As the brain functioning is more important than the correct answer, the person need not be able to physically select the correct answer through the response pad, but should be able to indicate that an answer has been identified to signal the end of the time period for the test task and begin the rest period and continue with the assessment. Thus, the person's pressing of the response pad may be used to control the progression of the assessment, and may be used to signal the start and stop periods of brain activity for use in identification of the BOLD signal from the fMRI scans.

Each of the various assessment protocols are carried out as follows:

Matrix Reasoning

Each test stimulus consists of a 3×3 matrix of figures, with a piece missing. Participants are instructed to deduce what the missing figure should be, and then select it from among the four choice alternatives presented on the right side of the matrix. An example matrix used in such an examination is shown in FIG. 8. The person is shown eight different FIGS. 74 and required to identify the missing figure, indicated at 78, by selecting from the four figures provided as answers and illustrated at 82.

Once the person has solved the problem, he indicates the answer by pushing a button on a fiber-optic response pad, which registered the amount of time it took to solve the problem, and advanced the computer to the next problem. Although the accuracy of the subject's responses was not tested, his response times were. The study's interest lies more within the brain functions engaged in trying to solve the problem, rather than whether the answer is correct or incorrect. This general principle applies to all six tasks.

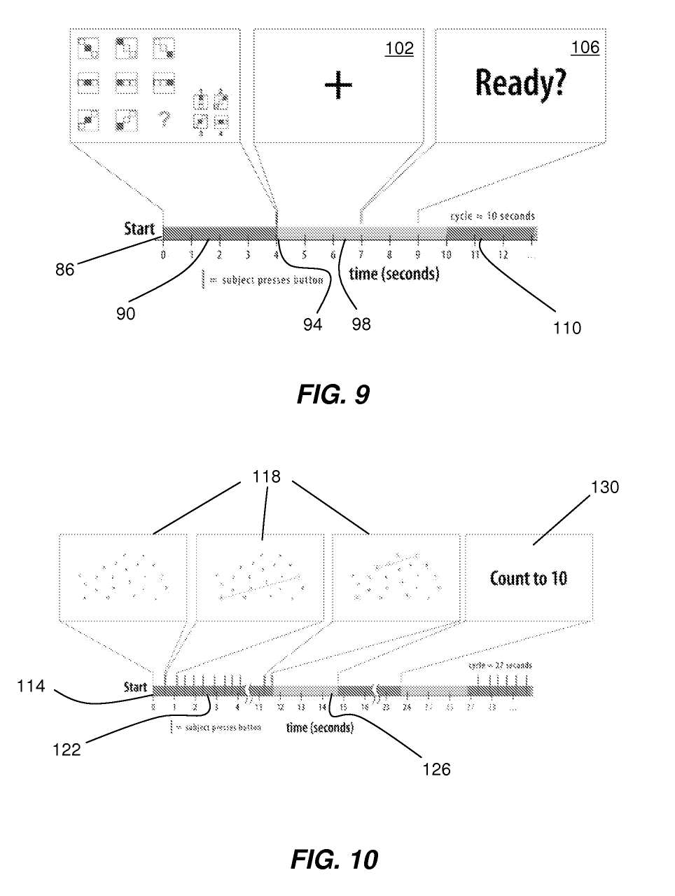

FIG. 9 further illustrates the timing and operation of the matrix reasoning assessment. The time line 86 shown in FIG. 9 illustrates how the person is presented with a problem and solves the problem during time period 90, the testing period. The person's selection of an answer 94 ends the testing period and starts the resting period 98, where a person may be shown a simple stimulus 102 to direct brain activity, and may then be shown a word or stimulus 106 to indicate that the next test is about to begin. Thereafter, a new testing period 110 begins with a new problem, and the testing cycle is repeated a desired number of times.

Trail Making Test

While in the fMRI scanner, the person is presented with a presentation of numbers and letters, with the first number in a sequence circled. The person is required to identify and find the subsequent numbers or letters in the sequence. As soon as the person found the next symbol in the sequence, he pushed a button on the fiber optic response pad, which then highlighted the next circled letter and drew a line connecting the two. If the subject were incorrect, this procedure allowed for a visual “correction” before permitting him to proceed.

FIG. 10 illustrates the time line 114 and additional aspects of the trail making test. The test involves presentation of the screen with numbers and letters and requires the person to select the proper symbols in sequence. Thus, different testing screens 118 are shown to a person throughout a testing period 122. The testing period 122 is thus divided into a series of smaller periods corresponding to each screen of the test. After a test trail is completed, a resting period 126 begins, which may involve counting to ten. Other trails may then be presented to the person during subsequent cycles of the test.

Face Recognition Test

Persons are shown groups of 15 faces while in the fMRI scanner, and are asked to remember each face. At the end of the scanning session, the person was shown another group of faces, both novel and previously viewed, and was asked to indicate which faces were ones that were seen previously while in the scanner. Accuracy for this post-test was recorded for each participant.

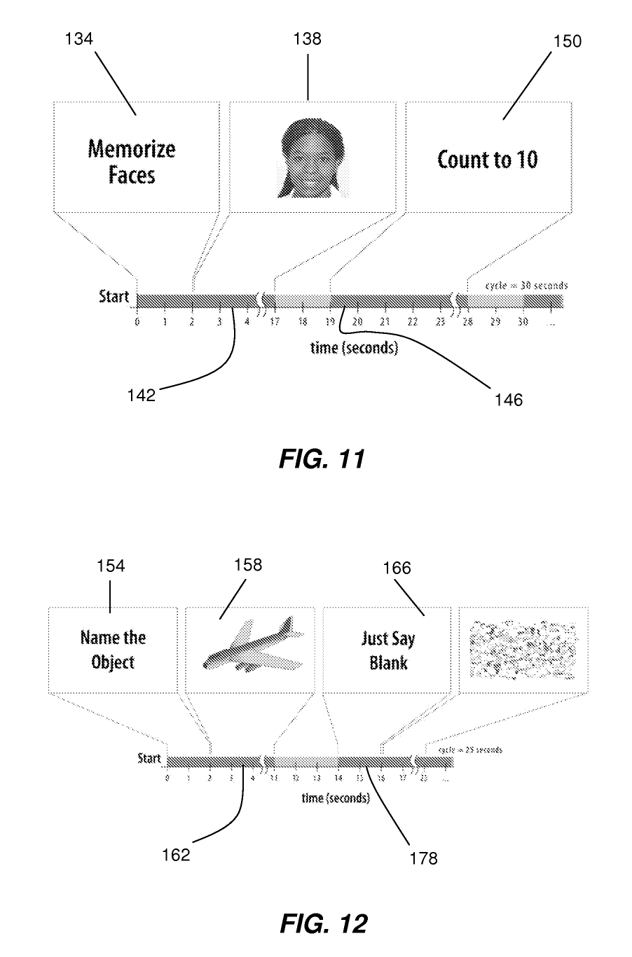

FIG. 11 shows the data encoding portion of the Face recognition test performed within the fMRI scanner. As illustrated, the test will typically present a screen 134 instructing a person to memorize the faces presented to them. Thereafter, faces are presented to the person, as indicated at 138. The faces 138 may be presented during a testing time period 142, and the person may then be presented with a rest period 146 and instructions 150, such as to count to 10.

Picture Naming Test

A person in the fMRI scanner is presented with a series of line drawings and is asked to silently name each picture, as vocalization while in the scanner produces motion artifacts which may distort the imaging. FIG. 12 illustrates the testing time table and protocol. The person may be presented with an instruction screen and then presented with picture screens during individual testing periods. The person is presented with resting screens 166 which control activity during the resting period 170, such as by directing the person to identify the screen as blank. The test thereafter cycles through additional pictures and rest periods.

Verbal Fluency Test

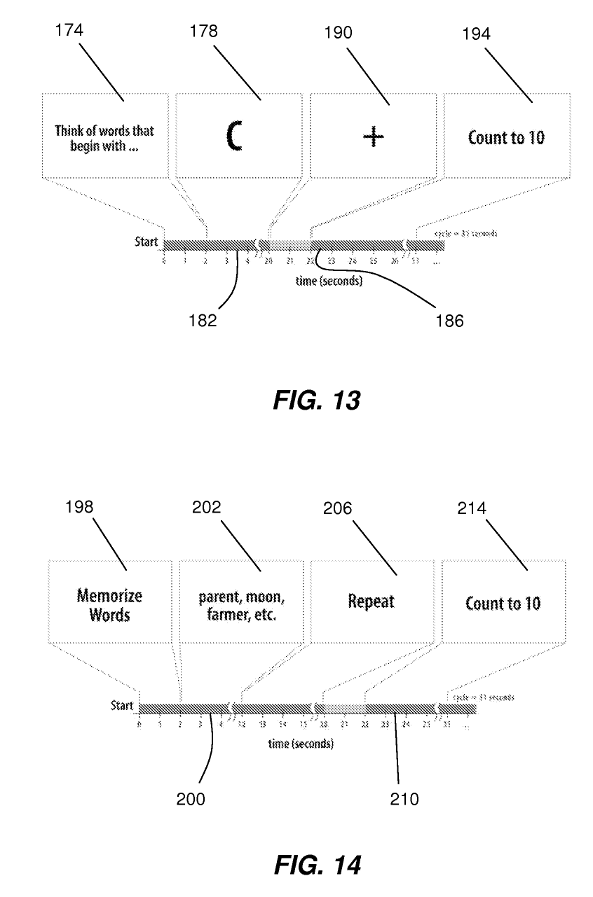

While in the scanner, the person is prompted with a screen that contains specific instructions such as: “Please silently think of as many words that begin with the letter . . . . ” A letter then appeared and the person silently thinks of as many words as he can that began with that letter. The following eight letters are used: F, A, S, C, L, P, R, and W. FIG. 13 shows the timing of the assessment protocol. The instructions 174 are first presented, and the presentation of a letter 178 begins the test period 182 of 22 seconds. After the test period 182, a rest period 186 begins, which may include simple symbols 190 to look at and directions to count 194 or perform another minimally difficult task to control the activities during the test period. The above test sequence is thereafter repeated to complete the tested letters.

Verbal Working Memory

As illustrated by FIG. 14, persons in the scanner are instructed to memorize a series of words 198 and, in a testing period 200, are presented a list of words one by one in a series of screens 202. At the end of the list, the person was prompted to silently recall or repeat 206 as many words as he could. In a resting period 210 the person is directed to count to ten or perform another simple controlled task. This process is repeated 5 times with different series of words.

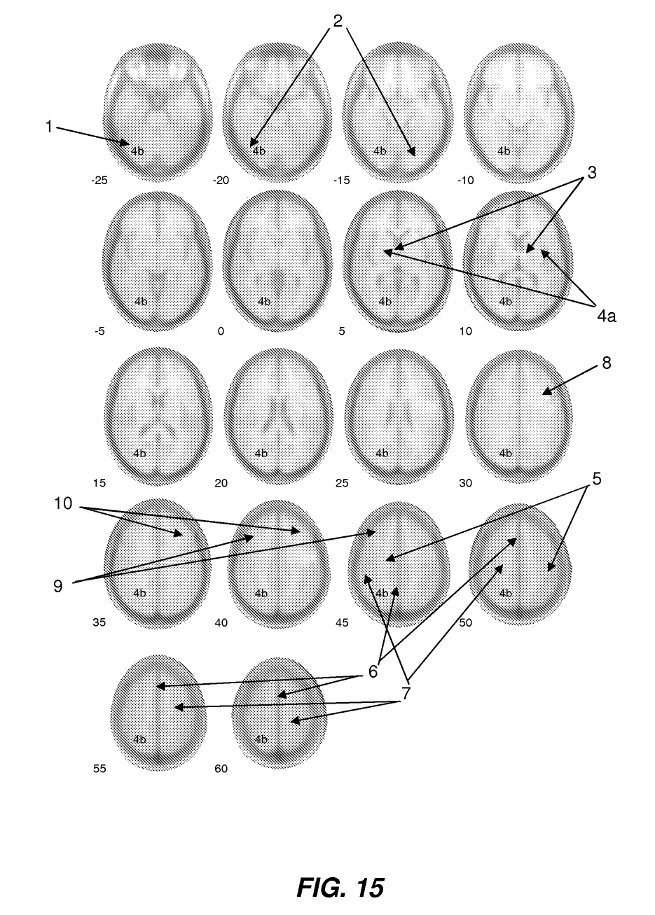

According to medical understanding of which areas of the brain are activated when performing different types of tasks, it is anticipated that the fMRI assessment protocols will generate activity in the following areas of the brain for each type of assessment. FIGS. 15-26 show the relevant cross sections of the brain, illustrating the predicted and measured brain response to the assessment protocols.

For the matrix reasoning test, it was anticipated that the following areas of the brain would show heightened activity, as illustrated in FIG. 15:
1. Bilateral inferior lateral occipital and inferior middle lateral occipital, stronger on the right
2. Bilateral fusiform gyrus
3. Bilateral thalamus (including inferior and superior)
4. (a) Bilateral frontal operculum (including the basal ganglia, bilaterally)
   (b) Dorsal processing stream (extending dorsolaterally from the primary visual areas in the occipital cortex)
5. Superior parietal lobe including inferior parietal sulci, stronger on the right
6. Dorsal anterior cingulated and bilateral medial supplementary motor areas, stronger on the left
7. Bilateral precentral gyrus activation, stronger on the left, medial frontal gyrus activation on the right
8. Anterior middle frontal gyrus activation on the left
9. Fronto-polar activation on the right
10. Tip of the superior frontal gyrus on the left

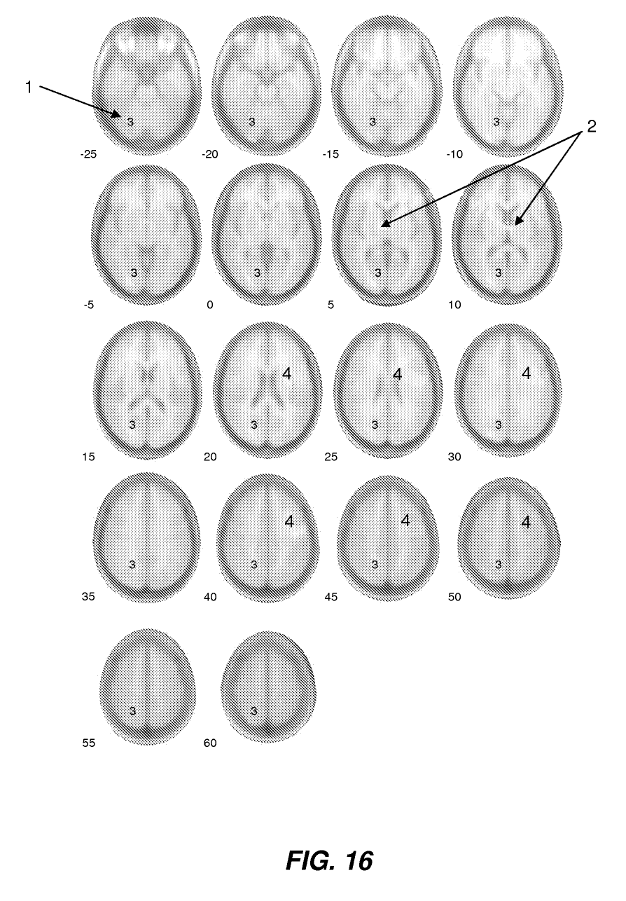

For the trail making test, it was expected that the following areas of the brain would show heightened activity, as illustrated in FIG. 16:
1. Bilateral fusiform, stronger on the left
2. Inferior thalamus, bilaterally
3. Dorsal processing stream from the lateral occipital cortex (LOC) to the superior parietal lobule, including the interparietal sulcus (IPS) bilaterally but stronger to the left
4. Left precentral motor cortex, left medial supplementary motor area (SMA)

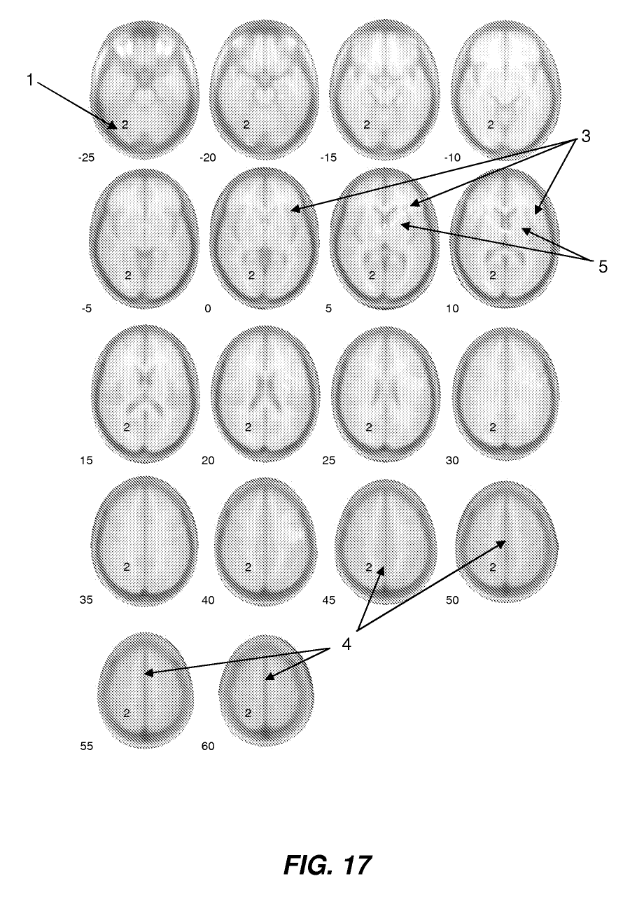

For the picture naming test, it was expected that the following areas of the brain would show heightened activity, as illustrated in FIG. 17:
1. Bilateral fusiform activation, much stronger on the right
2. Small dorsal processing stream activation, bilaterally
3. Strong left frontal operculum and left speech motor areas (at lower thresholds)
4. Bilateral anterior cingulated
5. Left thalamus activation

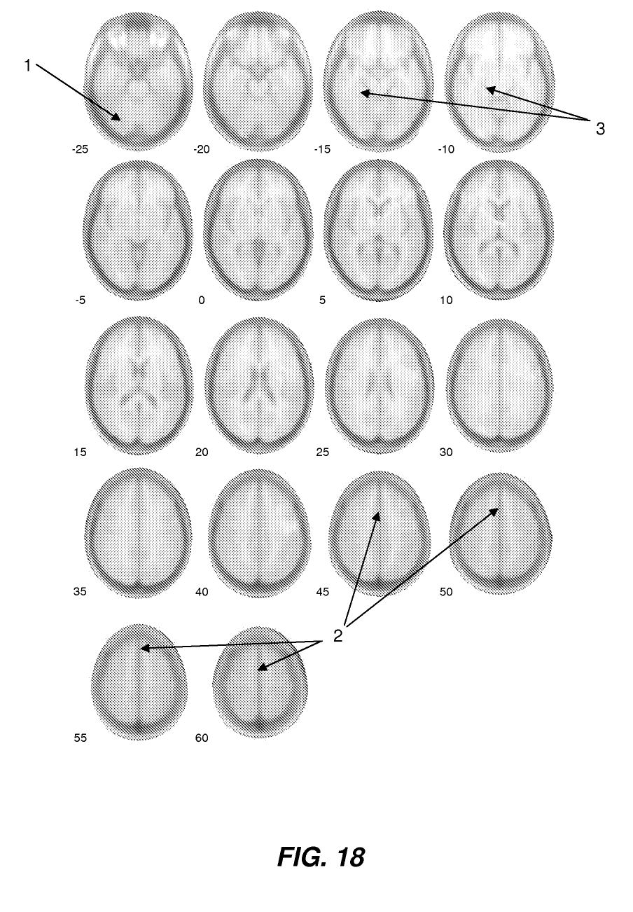

For the face recognition test, it was expected that the following areas of the brain would show heightened activity, as illustrated in FIG. 18:
1. Bilateral fusiform and LOC, much stronger activation on the right
2. SMA bilaterally, with stronger activation in the right precentral motor cortex, left caudate (superior portion)
3. Right posterior hippocampal, medial hippocampal, and parahippocampal (gyral activation)

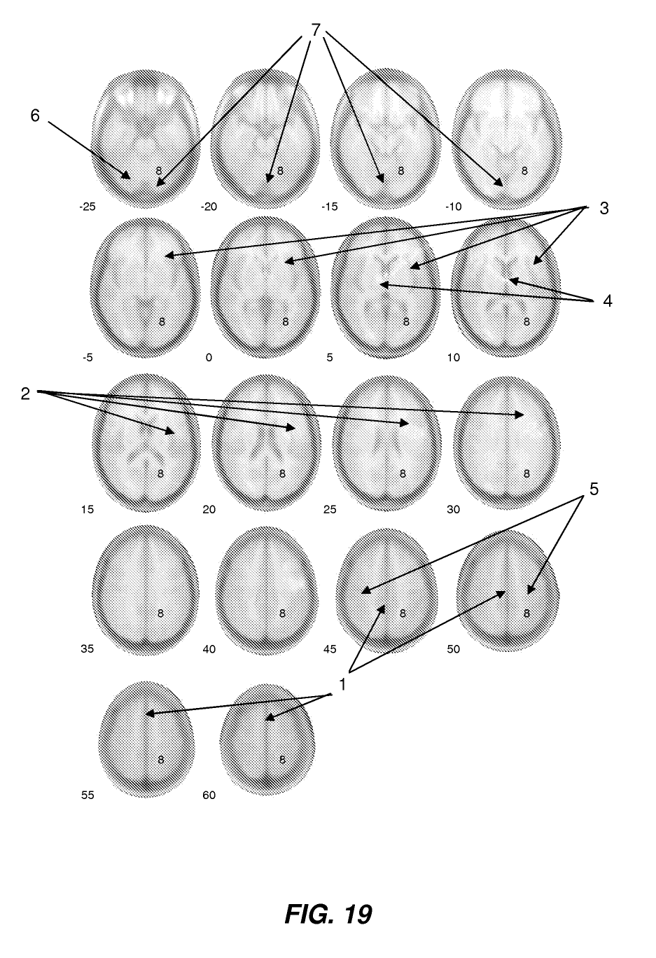

For the verbal memory test, it was expected that the following areas of the brain would show heightened activity, as illustrated in FIG. 19:
1. SMA bilaterally
2. Medial frontal gyrus
3. Left portions of the speech motor areas
4. Thalamus
5. IPS
6. Fusiform gyrus
7. Primary visual cortex (VI)
8. Some activation of the right inferior LOC

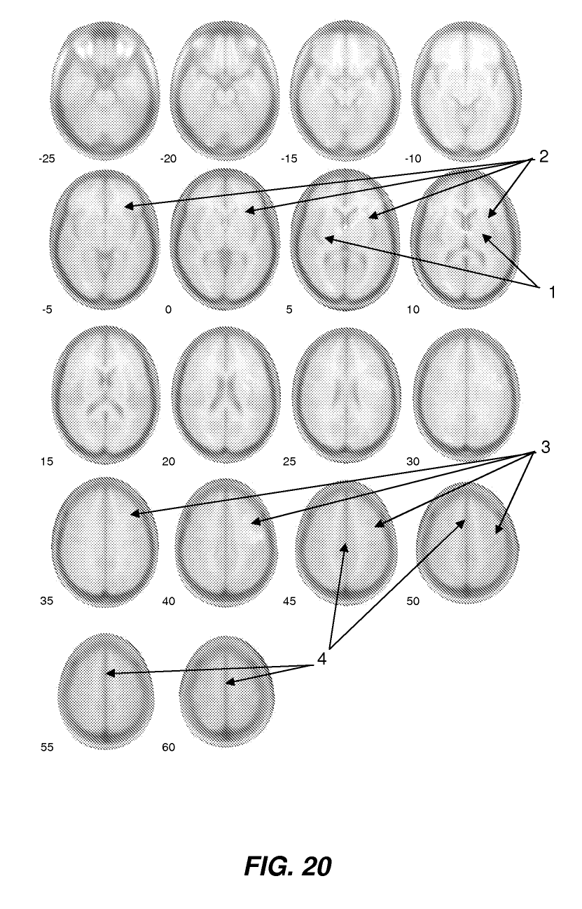

For the verbal fluency test, it was expected that the following areas of the brain would show heightened activity, as illustrated in FIG. 20:
1. Left caudate and left frontal operculum
2. Broca's area (left inferior frontal gyrus which extends to Broca's area)
3. Speech motor cortex and bilateral SMA
4. AC activity (greatest amount of the six tests)

A number of participants were used to initially test the effectiveness of the fMRI assessment protocols discussed herein and to create a data set corresponding to the brain response of average unimpaired individuals. The collective data from a group of participants is used to create a statistical mean of brain activation for the present fMRI assessment protocols and used to then assess the brain activity of a particular patient, as will be discusses herein.

In each scanning session, whole brain images were continuously collected every 2 seconds. A functional scanning protocol was used that was sensitive to blood oxygenation level dependent (BOLD) effects. In order to do this, a gradient echo, echoplanar imaging sequence was used. The specific timing parameters used were TE=30 ms TR=2000. 23 Axial slices with 5 mm thickness and no gap were used.

Data Analysis

The fMRI data was processed and analyzed using a sequence of commercial fMRI analysis software packages. The fMRI activation was assessed for two dimensions for each cognitive test: location(s) of activity foci in the brain, and the intensity of the activation change(s) for each locus. Group average activation probability maps were constructed from the individual activation data for each participant using Matlab software.

The fMRI Data was analyzed with statistical parametric mapping according to the following procedure: Prior to statistical analysis, the first four volumes of each run were discarded in order to avoid T-1 relaxation effects, and the remaining functional volumes from each person were realigned and resliced with sinc interpolation, using the first volume in the sequence as a reference. Realigned images were motion-corrected and unwarped according to residual motion-related signal changes that were calculated for three rigid translation and three rotation directions. The six motion-correction parameter vectors were then saved for use as confounding covariates in the statistical analysis. A mean image for each person was created using the realigned and motion-corrected volumes. A T-1 weighted SPGR anatomical MRI was coregistered to this mean image, ensuring that the functional and structural images were spatially aligned.

The structural image for each person was superimposed with the functional activation “hot spots” for display inspection purposes. The images were then spatially normalized to a brain template provided by the Montreal Neurological Institute (MNI), which is a composite of 305 brain samples. This step was necessary in order to place each participant's brain within a common stereotactic space.

Finally, the functional images were spatially smoothed with an 8 mm full width at half maximum (FWHM) isotropic Gaussian kernel. Smoothing is necessary in order to create average images across many persons and as a necessary condition for the application of Gaussian random field theory in group-level statistical inference.

A boxcar waveform convolved with a synthetic hemodynamic response function (HRF) with a 4 second lag-to-peak is used as a reference waveform for each condition. The data are high-passed-filtered using a set of discrete cosine basis functions with a cut-off period of 128 seconds. The synthetic HRF curve may be used to identify the scanning time where a peak response to an assessment task should occur. To condition temporal autocorrelations in the data, an AR1 correction was applied to each person's data during statistical analysis.

The resulting foci of activation (the activated hot spots in the brain corresponding to the assessment tests) are characterized in terms of peak height and spatial extent. The significance level (p<0.5) of each region was estimated and corrected for multiple contrasts using distributional approximations from the theory of Gaussian fields, both in terms of the probability that a region of the observed number of voxels could have occurred by chance, and the peak height observed could have occurred by chance, over the entire volume analyzed. Regions of significant activation were displayed as “hot spots” in terms of the resulting t statistic for each voxel that exceeded the significance threshold. After statistics were computed for each person, a group-level random effects model (RFX) was applied. The major benefit of using an RFX model is that only areas with activation consistent in all persons were able to survive the statistical analysis.

Because of the number of persons, the anatomical image and superimposed functional activation map for each person was displayed and compared individually, before averaging the scans together, in order to detect any gross deviations or outliers (i.e. a person without any activation/corrupted data). This strategy avoids sources of error that typically arise when anatomical and functional images are averaged across persons. Across-person averaging is often necessary in order to improve the signal-to-noise ratio of functional scans from lower quality MRI magnets.

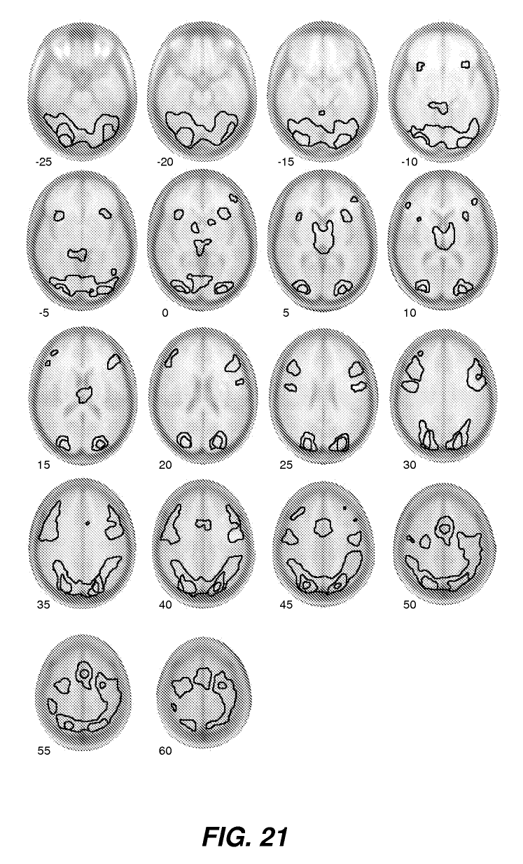

The average brain activation response to the present fMRI assessment protocols from a group of normal (unimpaired) individuals becomes a reference data set for comparison with individual patients being tested for an impairment. The mean activation for all persons included in the data set representing a normal brain response to the assessment protocols are shown in FIGS. 21-26 for the various testing protocols.

Matrix Reasoning

FIG. 21 illustrates the normal brain activation for the matrix reasoning tests. The activated areas of the brain include the bilateral fusiform gyri, with stronger activation on the right, bilateral inferior lateral occipital, and inferior middle lateral occipital, both stronger on the right, bilateral thalamus (including inferior and superior thalamus), bilateral frontal operculum (including the basal ganglia, bilaterally), the dorsal processing stream, which extends dorsolaterally from the primary visual areas in the occipital cortex to superior parietal lobule (SPL), including the inferior parietal sulci (IPS). Although this activation was bilateral, it was stronger on the right than the left. There was also activation in the dorsal anterior cingulate and bilateral medial supplementary motor areas (SMA), although it was stronger on the left. There is also bilateral precentral gyrus activation, although it was stronger on the left, medial frontal gyrus activation only on the right, and anterior middle frontal gyrus activation only on the left. There was also some evidence of small right fronto-polar activation on the right, and on the tip of the superior frontal gyrus on the left.

Trail Making Test

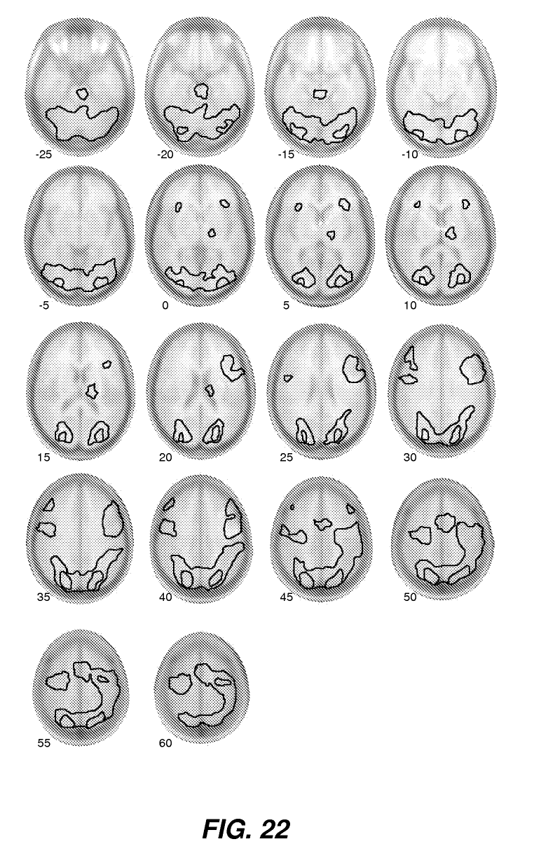

FIG. 22 illustrates the areas of brain activation during the present fMRI trail making test. The mean activation is almost identical to the matrix task, and included the bilateral fusiform, with stronger activation on the left, small activation in the inferior thalamus, bilaterally, the dorsal processing stream from the LOC to the superior parietal lobule, including the IPS, bilaterally, but with a little stronger extension into the IPS on the left. There was also activation in the left precentral motor cortex, and the left medial SMA.

Picture Naming Test

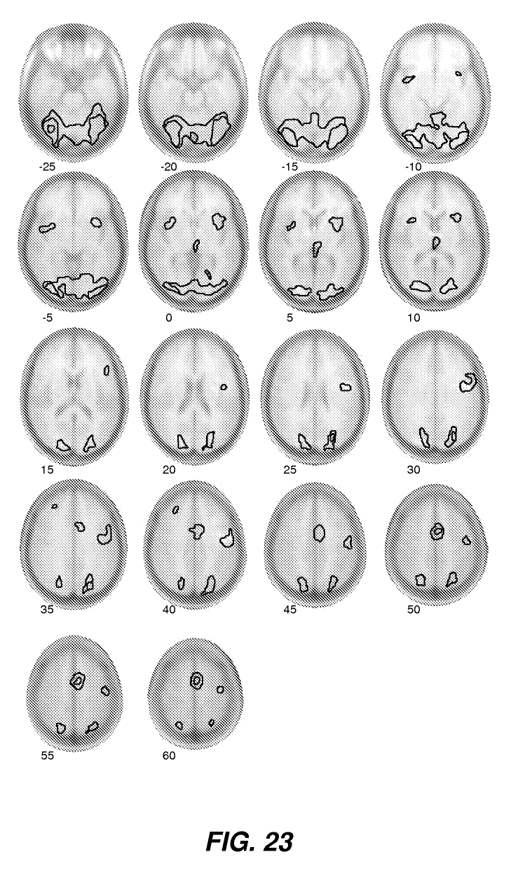

FIG. 23 illustrates the brain activity for the picture naming test of the present invention. The mean activation includes bilateral fusiform activation, but much stronger on the right, and some small dorsal processing stream activation, bilaterally. At lower thresholds, mean activation included areas which one would typically expect for a Picture Naming task, including strong left frontal operculum (Broca's area), bilateral anterior cingulate, left speech motor areas, and left thalamus activation. One may have expected to find frontal activation for this task, but due to the use of very common objects, (e.g. chair, airplane, etc.), the simplicity of the pictures would suggest less frontal lobe activation in controls. Pilot studies with dementia patients reveal higher activations in these areas for the same task, which suggests that those who are neurologically impaired require more effort and elicit greater activations for a simple task. It is anticipated that the use of more difficult pictures in the naming test will produce greater activations in these areas of the brain.

Face Recognition Test

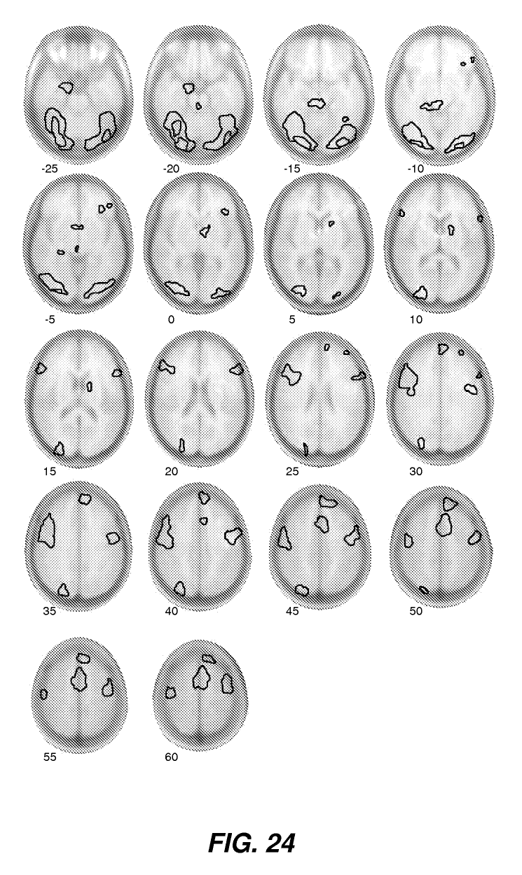

FIG. 24 illustrates the mean brain activity for the present face recognition test. The mean activation includes the SMA bilaterally, with stronger activation in the right precentral motor cortex, the left caudate (superior portion), bilateral fusiform and LOC, but with much stronger activation on the right. There was also right posterior hippocampal, medial hippocampal, and parahippocampal gyral activation.

Verbal Memory Test

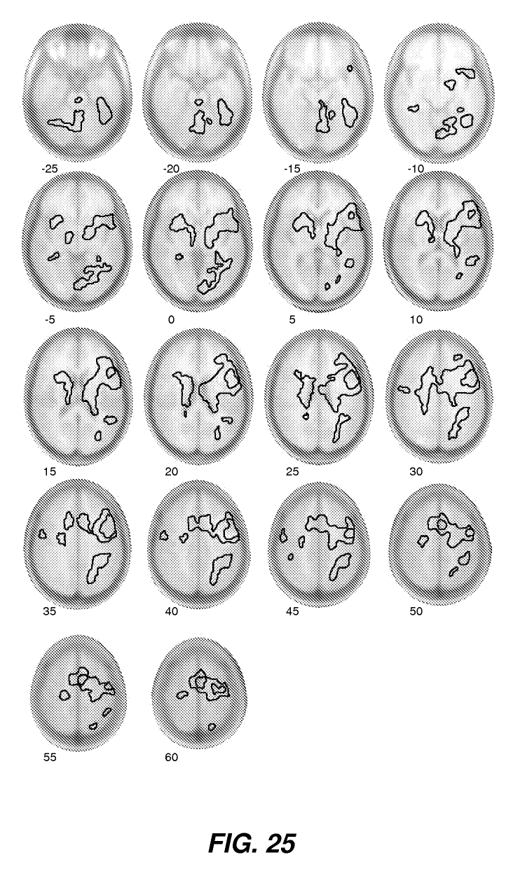

FIG. 25 illustrates the brain activity for the present verbal memory test. The mean activation includes the SMA bilaterally, with a very small activation in the medial frontal gyrus (MFG). There was activation on the left in the speech motor areas, the thalamus, the IPS, the fusiform, and the primary visual cortex (V1). There was also some activation on the right inferior LOC.

Verbal Fluency Test

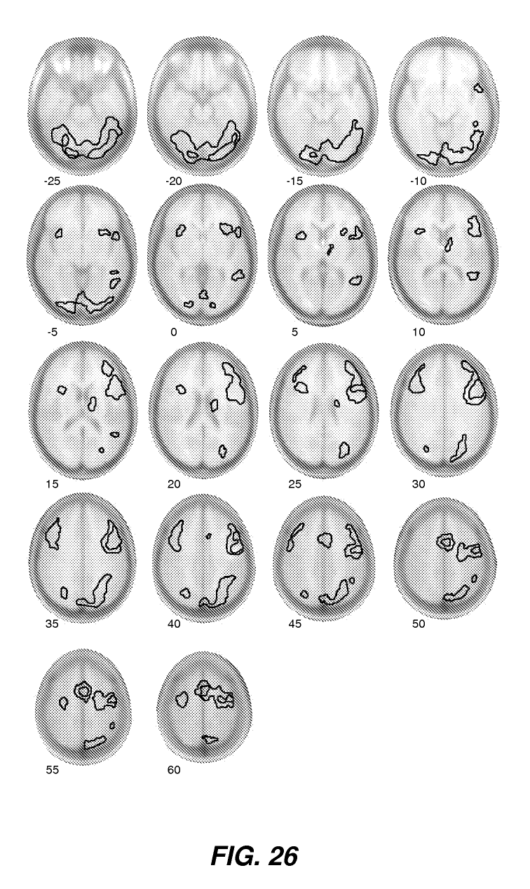

FIG. 26 illustrates the mean brain activity for the present verbal fluency test. The mean activation includes the left caudate and left frontal operculum, Broca's area (left inferior frontal gyrus which extends to Broca's area), the speech motor cortex, bilateral SMA. Also, out of all the six tasks, this task elicited the greater amount of AC activity. Also, there was no noted activation in visual areas.

In comparing 13 male versus 13 female brain activation results (fMRI scans) for each individual task, no statistical differences in activation between genders were found. In order to more confidently accept the null hypothesis of no sex differences, the data from all the experiments was examined using the most liberal voxel extent threshold that is scientifically acceptable (p<0.05, and cluster extent threshold of 0). Still no statistically significant differences were found. These findings suggest that current technology is not yet at a level that can easily differentiate between genders. It will also be appreciated that gender differences may be difficult to detect under the present assessment protocols because of the breadth of analysis. Because the present system for fMRI scanning during brain assessment protocols to detect impairment advantageously utilizes scanning of general areas of whole brain to detect brain activation for the assessment tasks, it is desirable to sacrifice some overall sensitivity for specific regions. For the available scanning and processing power of the fMRI systems, it is generally more desirable to scan the entire brain at a lower sensitivity than to scan smaller regions of the brain at higher sensitivity.

As fMRI scanning and analysis becomes more sensitive and able to detect smaller differences in brain activity, it is expected that it will be possible to more clearly and easily discover gender differences with regard to functional activation of the brain.

According to the present invention, patients may be evaluated using psychological assessments performed within an fMRI scanning environment to evaluate the patient for brain impairment or damage to the brain. Individual patients may be evaluated against a large data set of normal (unimpaired) individuals as has been discussed herein. Additionally, patients may be evaluated without the use of a large normative data set, such as by comparing before and after fMRI scans of the patient to evaluate potential brain injury of damage caused by an intervening event, the effectiveness of treatment, or the general changes in brain functionality as may be caused by degradation, healing, or the like.

In evaluating a patient, a logical approach would be to first divide the brain into functional regions, or regions of interest. Such may be identifying areas of the brain likely to have suffered injury or identifying areas of the brain which correspond to particular types of tasks or activities. Thereafter, one would be able to use any number of summary statistics across persons which could be plotted in that particular region of interest. For example, one could take the total number of persons for each region whose brain activation exceeded a certain threshold level. Alternatively, one could compute the distribution of the heights of activation peaks across persons. For example, if a T-value is set to a certain threshold, one could plot peak values which would equal a distribution that can be transformed into z-scores. Yet another variation and an additional option is to do an analysis of peak activations and the extent of activations within each of the regions of interest, in terms of how many voxels were activated in that particular area. The distribution of the extent of the voxels can then be converted to z-scores with standard deviations, etc. It is thus appreciated that various methods exist for evaluating a patient against a normative data set.

According to a method of using the present invention to evaluate a patient for brain impairment, the patient may be placed into a MRI scanner capable of performing the desired fMRI scanning. The person would then undergo the neuropsychological assessments described herein while fMRI scans are taken of the patient's brain. In evaluating the fMRI scans, the scans taken during the various tests (such as the scans representing the peak brain activation for the particular tasks) may be compared to the composite brain scans of the normative data set, as represented in FIGS. 21-26. It is appreciated that the normative data set compiled from a large number of unimpaired individuals represents a statistical average for brain activity while performing the assessment tasks.

The comparison between the patient and the normative data set scans may be performed in a variety of ways. Initially, a visual comparison may be performed to identify any readily apparent differences, i.e. to identify a portion of the patient's brain which presents clearly impaired functionality. Statistical analysis of the patient's scan data and the normative data may be performed. As discussed, the analysis may use a variety of different methods to determine if the patient presents impaired functionality compared to the normal population. The analysis may evaluate the peak signal intensity for the BOLD response, evaluate a combination of the peak and duration of the response (an integral, or area under the curve, approach), evaluate the extent of brain tissue achieving a threshold response, etc. in order to determine if the patient's brain response, as measured by the BOLD signals, falls within the normal distribution of the normal population or represents an impairment.

The present invention is useful in detecting and evaluating brain injuries which have been difficult to evaluate. For example, mild brain injuries (MTBI) are often undetected or overlooked due to the fact that a mild injury may not result in significant structural damage to the brain, and thus result in structurally intact static MRI images. Although an MTBI may be structurally intact as viewed in a MRI scan, there are many mild injuries that result in confusion, attention and concentration difficulties, disorientation, and difficulties with problem solving. The present invention thus provides a supplemental tool for diagnosis of mild injuries which are otherwise difficult to diagnose.

Similarly, the present invention may be useful in evaluating and assessing the present of post traumatic stress disorder (PTSD). Many individuals, such as fire fighters, police, soldiers, etc., are exposed to traumatic situations and afterwards complain of symptoms which may be caused by PTSD. The present invention may be used to detect abnormal brain functioning as compared to the normal population to detect PTSD.

It will also be appreciated that, in a similar way that the present invention constructed a data set of a normal (unimpaired) population for use in comparative diagnosis of impairment, a data set may be constructed using persons who are exposed to traumatic situations (such as fire fighters, police, etc.) to determine the specific regions of the brain where PTSD effects may be observed and to determine exactly how PTSD affects the brain functionality. Persons such as police and fire fighters may volunteer to perform scans before and after a traumatic event to create such a data set of PTSD changes. The data may useful in identifying one or more types of PTSD changes to brain functionality, and to create a normative data set of the changes. By scanning individuals who are regularly confronted with traumatic situations, (e.g. fire fighters, police, etc.), pre- and post-trauma, one may be able to more clearly isolate the effects of intense traumatic exposure on neurochemistry. The data set may be thus utilized to determine if a person has PTSD, another brain impairment not related to trauma, or no impairment. Such a determination may be quite important in determining what treatment is appropriate and who is financially responsible for the treatment and any disability benefits.

The comparison of brain activity of a person claiming an injury to the normative data sets of persons having a particular injury may be useful in identifying the type and extent of injury in the particular patient. The fMRI brain scans may be compared against the normative brain scan data for injured persons to compare, both visually and statistically, the extent of brain activity, the areas of impaired brain activity, etc. Such a comparison against normative data sets of injured persons may allow a better determination of both type and extent of injury for a patient.

Additionally, the present invention may be utilized for the successful diagnosing and assessing patterns of brain activation in a before and after scenario. For example, fMRI neuro-imaging during neuropsychological assessment tests according to the present invention before treatment could be compared to imaging for the same assessments after treatment, evaluating the success of therapy based on observed brain activations. The present invention may thus be used to verify the effectiveness of treatment, whether the treatment is passage of time, physical and emotional therapy, drugs, etc. Similarly, before and after brain imaging may be used to track the progression of a disease or condition which may impair brain functionality. The methodology and system of the present invention may also be used to examine the effect of drugs or the like on the brain, allowing physicians or researchers to determine if a drug is causing a desired effect within the brain, or determine if a drug is causing an undesired side effect and impairing brain functionality.

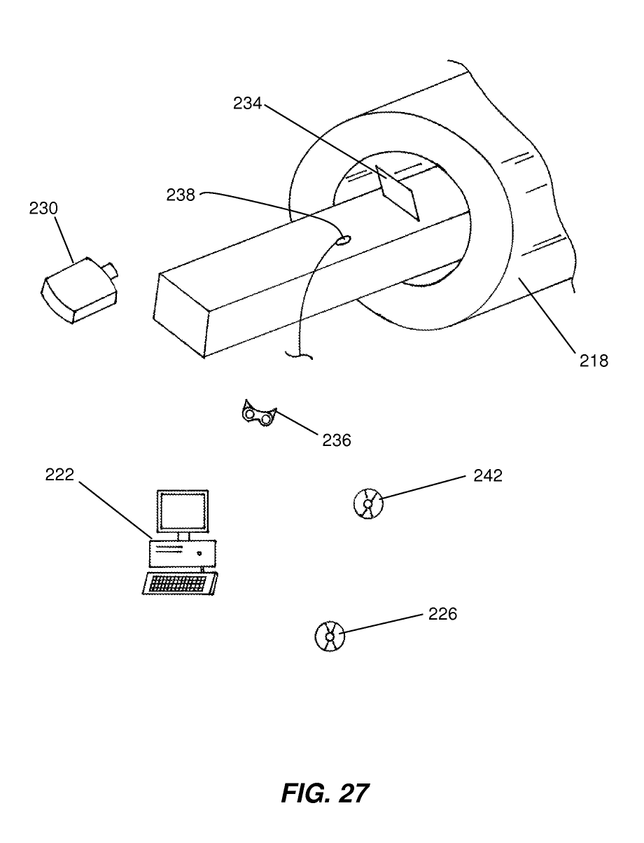

Turning now to FIG. 27, a system for performing analysis of brain functionality as discussed herein is shown. The system includes the various pieces necessary for fMRI scanning as discussed herein. It will be appreciated that the various elements discussed are exemplary and that suitable alternatives may be substituted therefore. According to the present invention, a system for fMRI imaging of a patient while undergoing the neuropsychological assessments of the present invention may include an fMRI scanner 218, a computer system 222 which may have the present assessment tests 226 stored therein in computerized form and having an interface for communication with the patient and other testing devices, a projector 230 and screen 234 for projecting the test screens and information onto a surface visible to the patient while in the fMRI scanner, a communications module 238, such as a hand piece 238, for allowing the patient to communicate with the computer and perform the assessment tasks, a normative data set 242 for evaluation of the patient fMRI results, and may include a computer or other necessary equipment for processing and evaluating data.

The various structures identified will perform the functions discussed above in performing an fMRI analysis of a patient. Thus, the assessment tests 226 comprise assessment tests suitable for use within an fMRI environment such as are discussed above.

It is understood that the fMRI scanning machines 218 utilize powerful magnets. As such, conventional devices such as a computer terminal may not be used to directly communicate with the patient. Metal objects are isolated from the scanning environment. Thus, the present invention utilizes display means which are capable of displaying the test screens to the patient without adversely affecting the fMRI scanning environment or being adversely affected by the fMRI environment. The display means may be a remotely located projector 230 and non-metallic screen 234 to present the test screens to the patient. The projector 230 would typically be located in an adjacent room where it could project images inside of the MRI machine 218 and the screen 234 would typically be located inside of the MRI machine where it is visible to the patient. The screen may be a glass or plastic screen which reflects the images from the projector to the patient in a manner similar to movie screens and projectors or teleprompters.

Alternatively, the display means may be a pair of LCD or fiber optic goggles 236 or other similar device. The goggles 236 would typically be made of non-metallic materials and would include a fiber optic connection to the computer 222 for operating the goggles 236 so as to not interfere with the fMRI environment. The goggles 236 thus transmit the desired images to the patient while the patient is within the MRI machine 218. The patient will have their head inside of the fMRI scanner, and should not move their head during the test. As such, the non-metallic screen 234 would be placed where it is easily visible to the patient and where a projector outside of the fMRI scanning environment can project the desired images onto the screen.

The patient will have a hand piece 238 to indicate that they have performed a certain task. Many different hand pieces may work, such as a fiber optic pad which transmits a touch signal to a sensor and then to the computer interface, or a squeeze bulb and tubing which transfers a pressure pulse to a pressure sensor outside of the fMRI scanning environment which transmits an electronic signal to the computer interface. These hand pieces 238 may be constructed out of plastic, rubber, glass, etc. so as to not adversely affect the fMRI scans or be affected by the magnetic fields. The patient will typically control the test by the particular hand piece 238 interfacing with the testing computer 222. As discussed, the test is performed by displaying images to a patient and requesting that the patient perform a mental task, such as determining the following object in a sequence. When the patient has performed the task, they will use the hand piece 238 to transmit a signal to the computer and advance the test.

The system will typically include a normative data 242 set of fMRI scans as has been discussed. The data set may be average brain responses for a normal, unimpaired population. The normative data set may also include average brain responses of persons having a particular type of brain injury so as to allow the patient's results to be analyzed against the data set to match impaired functionality to a particular injury which typically causes the particular type of impaired functionality. The data set 242 may also include previous test data for a particular patient so as to show changes in the particular patient's brain functionality due to trauma, treatment, passage of time, etc. The data set 242 may be used to diagnose and assess the severity of impairment, or to assess the effectiveness of treatment. Thus, multiple scanning sessions may be used to perform before and after scans of the patient to assess improvement or change or the lack thereof in the patient's condition. The normative data is important as it provides a background against which the scan information for a particular patient is analyzed, providing an objective analysis of the patient. Reducing or eliminating the subjectivity in mental performance is a significant advantage of the present invention.

The present invention is advantageous as it thus allows for direct measuring of brain functionality while performing categorical tasks that activate particular areas of the brain and as it provides for normative data to compare the test results against to identify impairment. Previous testing methods required a physician to observe a patient's performance at a task and thereby infer brain functionality, requiring a large amount of subjective evaluation by the physician. The present methodology and system does not require any measure of the actual performance of the patient, instead measuring the brain functionality while attempting the task.

The above described system allows the desired images forming the assessment test to be displayed to the patient while in the fMRI scanner. As has been discussed, the patient should not move or talk while in the scanner. As such, the testing system and assessment tests have been designed to allow the patient to perform the test without such movement. Because the present methods and system do not require any determination of the patient's actual performance, there is no need for a patient to write, speak, or otherwise record their test answers.

Conventional paper and pencil assessment tests require the patient to physically perform certain tasks, such as identifying objects or tracing out logical paths. An assessment of impairment is based on the patient's test performance. In comparison, the present invention does not require the patient to even select an answer for the assessment tests. The patient is shown an image for a particular phase of the test and is given a task to perform, such as identifying an object or path, but does not actually select the correct answer. Instead, the patient simply indicates that they have determined an answer. As such, the patient need not use an overly cumbersome interface, but may simply use a single output indicator such as a squeeze bulb or fiber optic touch pad. These devices indicate that the patient has completed a step of the test, but do not register any particular answer. Thus, the test does not require or encourage the patient to move, and is compatible with the fMRI environment.

The use of a patient output device 238 such as a squeeze bulb or fiber optic pad also allows for more accurate correlation of the BOLD response illustrated in the fMRI scans with the brain activity corresponding to that response. As discussed, the patient will signal the completion of an assessment task by pressing on a pad or squeeze bulb. The patient output may be used to identify the scan times which correspond to the assessment task. For any particular assessment task, the start of the neural activity will roughly correspond to the time where the task image is initially presented to the patient. The end of the neural activity will typically correspond to the patient using the output device to signal that an answer has been found. Since the period of peak BOLD signal typically follows the period of neural activity with about a five second delay, the task start time (first showing the image to the patient) and the task end time (patient pressing the output device) may be used to determine the relevant period of fMRI scans by correlating the timing of the test events (presenting assessment images, patient output, etc.) with the scan times and using these test events to identify the relevant scans.

Additionally, the test controls what the patient does between steps of any particular test, and provides controlled resting periods. As the fMRI environment is a more tiresome and stressful environment (the person can not talk or move, there is noise, etc.), the controlled resting periods (such as counting) and control of what the patient does between steps or portions of the assessment test helps to keep the patient from becoming overwhelmed or tired and also helps to keep the patient from performing undesired tasks such as daydreaming between test tasks. Both of these improve the accuracy of the test. As discussed, the BOLD signal is time dependent and can be more accurately measured and analyzed for a particular brain activity if the timing of that activity is controlled and if the activity has more distinct start and stop periods and is not immediately preceded or followed by other brain activities. Thus, the present invention provides a testing system which allows an assessment test to be administered to a patient while in an fMRI environment, provides a normative background against which the individual results may be compared, and provides testing protocols which control patient brain activity during testing periods and non testing periods, and which allow for accurate scanning and identification of the brain activity corresponding to a particular assessment task.

There is thus disclosed an improved method and system for assessing brain function using functional magnetic resonance imaging. It will be appreciated that numerous changes may be made to the present invention without departing from the scope of the claims.

What is claimed is:

1. A system for detecting and assessing brain impairment comprising:
a neuropsychological assessment test, the assessment test comprising a series of images which require a person to perform a particular type of task;
a display device configured for displaying image screens of the assessment test to a patient while the patient is in an MRI scanner such that the assessment test is performed while fMRI scans are taken of the patient's brain;
an output device configured for use by a patient while fMRI scans are taken of the patient's brain, the output device being connected to the computer and configured for controlling the computer so as to allow the patient to take the assessment test; and
a normative data set comprising fMRI scanning images from a number of individuals, said fMRI scanning images being taken while each of said number of individuals are taking said assessment test within a fMRI environment.

2. The system of claim 1, further comprising a computer having the neuropsychological assessment test stored thereon in electronic format.

3. The system of claim 1, wherein the output device comprises a hand piece, and wherein said hand piece is configured for providing a signal to indicate that a patient has performed a task.

4. The system of claim 1, wherein the output device transmits a signal to indicate that a patient has completed a portion of the assessment test without indicating the result of the portion of the assessment test.

5. The system of claim 1, wherein the display device comprises a projector configured for projecting image screens of the assessment test and a screen disposed so as to be visible to a patient in the MRI scanner while fMRI scans are taken of the patient's brain.

6. The system of claim 1, wherein the display device comprises goggles.

7. The system of claim 1, further comprising an fMRI scanner.

8. The system of claim 1, wherein the assessment test comprises images configured for requiring a patient to perform a category of task selected from the group of: matrix reasoning, trail making, face recognition, picture naming, verbal fluency, and verbal working memory.

9. The system of claim 1, wherein the assessment test comprises a visual instruction to a patient to perform a particular task, a visual indication of the task to be performed, and a visual indication to the patient to control the patient activity after performing the task.

10. The system of claim 1, wherein the system is configured for detecting a patient's Blood Oxygen Level Dependent (BOLD) brain signal corresponding to the performance of predetermined tasks and comparing the BOLD signal to a normative data set of BOLD signals to thereby determine brain impairment in the patient.

11. The system of claim 1, wherein the output device is configured for providing a signal to a computer to identify the times during which the patient was thinking about an assessment test task.

12. The system of claim 11, wherein the times during which the patient was thinking about an assessment test task are correlated to the fMRI scan times to thereby identify the fMRI scans which show brain activity corresponding to the particular assessment task.

13. The system of claim 12, wherein the fMRI scans show Blood Oxygen Level Dependent brain signals.

14. A method for detecting and assessing brain impairment comprising:
placing a patient in an fMRI scanning environment so as to facilitate fMRI scans of the patient's brain;
having the patient perform a neuropsychological assessment test while in the fMRI scanning environment;
operating the fMRI scanner so as to capture fMRI scans of the patient's brain while the patient is performing the neuropsychological assessment test so as to detect brain signals from the patient's brain to thereby detect brain functionality during the neuropsychological assessment; and
comparing the fMRI scans of the patient's brain with a data set of fMRI scans to thereby detect statistical differences between the patient's fMRI scans and the data set of fMRI scans and thereby detect brain impairment in the patient, said data set of fMRI scans comprising brain signals of a group of individuals performing the same neuropsychological assessment while within an fMRI scanning environment.

15. The method of claim 14, wherein the brain signals comprise Blood Oxygen Level Dependent (BOLD) signals.

16. The method of claim 14, wherein the step of having the patient perform a neuropsychological assessment comprises, more specifically:
directing a patient to perform a desired task;
presenting an image to a patient so as to facilitate the performance of said task; and
the patient using an output device to indicate that the task has been performed without indicating the outcome of the task.

17. The method of claim 16, wherein the step of having the patient perform a neuropsychological assessment further comprises:
directing the patient to perform a resting task;
presenting an image to the patient so as to facilitate the performance of a second desired task; and
the patient using an output device to indicate that the task has been performed without indicating the outcome of the task.

18. The method of claim 14, wherein the assessment test comprises categories of tasks selected from the group consisting of: matrix reasoning, trail making, face recognition, picture naming, verbal fluency, and verbal working memory.

19. The method of claim 14, wherein the method comprises projecting images which comprise portions of the assessment test to a screen within a MRI machine such that the images are visible to a patient who is in the MRI machine such that the patient can perform the task which is indicated by the projected image while in the MRI machine.

20. The method of claim 14, wherein the step of having the patient perform a neuropsychological assessment test while in the fMRI scanning environment comprises:
projecting an image to the patient which provides instructions to the patient about a task which is to be performed;
projecting an image to the patient which gives the patient a task to be performed in accordance with the instructions;
the patient mentally performing the task; and
the patient indicating that they have completed the task by using an output device to signal to a computer that the task has been completed.

21. The method of claim 20, wherein the method further comprises:
projecting an image to the patient to direct the patient's thoughts during a resting phase;
projecting an image to the patient which provides instructions to the patient about a second task which is to be performed;
projecting an image to the patient which gives the patient a second task to be performed in accordance with the instructions;
the patient mentally performing the second task; and
the patient indicating that they have completed the second task by using an output device to signal to a computer that the task has been completed.

22. The method of claim 21, wherein the method further comprises:
in said fMRI scanning environment, an MRI machine taking fMRI images of the patient's brain while the patient performs the assessment test;
a computer recording the fMRI images;
the output device transmitting a signal to the computer to signal that the patient has completed a task;
the computer recording the signal from the output device; and
the computer correlating the signal from the output device to the fMRI scans to thereby identify the fMRI scans which correspond to brain activity which is expected to occur during a portion of the assessment test.
